(19) United States
(12) Patent Application Publication
(10) Pub. No.: US 2023/0027819 A1
(43) Pub. Date: Jan. 26, 2023

(54) CELL-PENETRATING PEPTIDE DIMERS, METHOD FOR PREPARING THE SAME, AND CARGO DELIVERY SYSTEM USING THE SAME

(71) Applicants: UPPTHERA, Incheon (KR); SEOUL NATIONAL UNIVERSITY R&DB FOUNDATION, Seoul (KR)

(72) Inventors: Tai Hyun PARK, Seoul (KR); Haein LEE, Seongnam-si (KR); Yeong Kyu PARK, Seoul (KR); SooHee RYU, Incheon (KR); Seong Hoon KIM, Incheon (KR); Hyeong Seok LEE, Incheon (KR)

(21) Appl. No.: 17/899,634

(22) Filed: Aug. 31, 2022

Related U.S. Application Data
(63) Continuation of application No. 17/244,973, filed on Apr. 30, 2021.
Application No. 17/244,973 is a continuation of application No. PCT/KR2021/003651, filed on Mar. 24, 2021.

(30) Foreign Application Priority Data
Mar. 25, 2020 (KR) 10-2020-0036503
May 15, 2020 (KR) PCT/KR2020/006430
Sep. 11, 2020 (KR) 10-2020-0116600

(51) Int. Cl. C07K 14/47; B82Y 5/00

(52) U.S. Cl. CPC C07K 14/47; B82Y 5/00; C07K 2319/00

(57) ABSTRACT

The present invention relates to a cell-penetrating peptide dimer comprising: a first peptide domain consisting of the amino acid sequence of SEQ ID NO: 1; a second 30Kc19α peptide domain consisting of the amino acid sequence of SEQ ID NO: 1; and a peptide linker connecting the first and second peptide domains, a method for preparing the peptide dimer, a cargo delivery system in which a cargo is conjugated to the dimer; and a use thereof. The cell-penetrating peptide dimer according to the present invention may have excellent cell-penetrating properties, thereby being usefully employed as the cargo delivery system.

### CROSS-REFERENCE TO RELATED APPLICATION

This application is a Continuation application of U.S. patent application Ser. No. 17/244,973, filed on Apr. 30, 2021, which is a Bypass Continuation application of International Application No. PCT/KR2021/003651, filed on Mar. 24, 2021 and designating the United States, the International Application claiming a priority date of Mar. 25, 2020 based on prior Korean Patent Application No. 10-2020-0036503, filed on Mar. 25, 2020 and claiming a priority based on prior Korean Patent Application No. 10-2020-0116600, filed on Sep. 11, 2020 and claiming a priority based on prior International Application No. PCT/KR2020/006430, filed on May 15, 2020. The disclosure of which is incorporated herein by reference in its entirety.

### SEQUENCE LISTING

The instant application contains a Sequence Listing which has been submitted electronically in XML format and is hereby incorporated by reference in its entirety. The Sequence Listing, created on Aug. 30, 2022, is 14,258 bytes in size and is named NP_PFP210010US_CA_sequence_listing.XML.

### BACKGROUND

### 1. Field of the Invention

The present invention relates to a cell-penetrating peptide dimer, a method for preparing the same, and an intracellular delivery system using the same.

### 2. Discussion of Related Art

Peptides, proteins, nucleic acids, and the like, have great potential as therapeutic substances, but have limited use since these substances are not able to penetrate cell membranes of target cells. In addition, in many cases, even small molecule compounds are often unable to penetrate a lipid bilayer of cell membranes due to properties or structures thereof. Accordingly, there have been attempts to move the therapeutic substance into cells by electroporation, a membrane fusion method using liposomes, heat shock, or the like. However, there are many restrictions in moving the substances into the cells while maintaining the activity of the substances simultaneously without damaging the cell membranes.

In this situation, cell-penetrating proteins or peptides have recently attracted attention. Among these, a trans-activator of transcription (TAT) protein derived from human immunodeficiency virus-1 (HIV-1) has been studied the most. It is known that a peptide consisting of 47th to 57th amino acids in the TAT protein consisting of 86 amino acids has a cell-penetrating function. Similarly, it is known that amino acids 267 to 300 of the VP22 protein of HSV-1, amino acids 339 to 355 of the Antennapedia (Antp) of a drosophila, artificially synthesized positively charged peptides, and the like, function as cell-penetrating peptides. Based on these facts, studies have been conducted in which cargo substances such as proteins or nucleic acids are bound to cell-penetrating peptides and delivered into cells (Guidotti, Giulia, Liliana Brambilla, and Daniela Rossi. Trends in pharmacological sciences 38.4 (2017): 406-424).

The 30Kc19 protein is a protein having a size of about 28 kDa derived from the hemolymph of silkworm (Bombyx mori) and has been applied to various studies since this protein has anti-apoptotic activity, protein stabilization or solubility enhancement function, and the like. It is known that the 30Kc19 protein is also a cell-penetrating peptide having a cell-penetrating function (Korean Patent Laid-Open No. 10-2011-0003889).

The mechanism of cell-penetration of the 30Kc19 protein has not been clearly elucidated, but a tendency to form a 30Kc19 protein dimer in the presence of an amphiphilic substance such as sodium dodecyl sulfate or phospholipid has been reported (see Park, Hee Ho, et al. Biotechnology journal 9.12 (2014): 1582-1593).

Meanwhile, the structure of the 30Kc19 protein is largely divided into 30Kc19α which is an N-terminal domain of an alpha-helix structure, and 30Kc19β which is a C-terminal domain of a beta-folding structure. Among these, it is known that the 30Kc19α is a domain having a cell-penetration function (see Ryu, Jina, et al. Biotechnology journal 11.11 (2016): 1443-1451). In addition, it has been disclosed that a cargo material may be attached to the recombinant 30Kc19α domain to be used as a cargo delivery system (Korean Patent Registration No. 10-1626343).

### SUMMARY OF THE INVENTION

An object of the present invention is to provide a peptide dimer with improved cell-penetrating properties.

Another object of the present invention is to provide a method for preparing the peptide dimer.

Still another object of the present invention is to provide a cargo delivery system using the peptide dimer and a use thereof.

The present inventors confirmed that a peptide dimer in which two peptide domains each consisting of the amino acid sequence of SEQ ID NO: 1 are connected to each other by a peptide linker had remarkably excellent cell-penetration efficiency, and completed the present invention (FIG. 1). Accordingly, the present invention provides a cell-penetrating peptide dimer, a method for preparing the same, a cargo delivery system using the same, and a use thereof.

Cell-Penetrating Peptide Dimer

In one general aspect, the present invention provides a novel cell-penetrating peptide dimer comprising: a first peptide domain consisting of the amino acid sequence of SEQ ID NO: 1; a second peptide domain consisting of the amino acid sequence of SEQ ID NO: 1; and a peptide linker connecting the first and second peptide domains, wherein the cell-penetrating peptide dimer is a fusion protein, and the peptide linker connects a carboxyl-terminus of the first peptide domain and an amino-terminus of the second peptide domain to each other, and consists of 100 or less amino acids.

The novel cell-penetrating peptide dimer provided by the present invention is characterized in that the peptide domains (first and second peptide domains), each of which is a protein consisting of 88 amino acids, are connected to each other by a peptide linker consisting of 100 or less amino acids (FIG. 1). In the field of protein engineering to which the present invention pertains, additional protein modification methods such as short-length peptides (for example, target peptides) or glycosylation (for example, PEGylation) without changing the sequence of the novel protein are known. In consideration of the number of amino acids and molecular weight of each peptide domain configuring the peptide dimer of the present invention, it may be anticipated that even if the conventional protein modification method in the art is additionally applied to the novel cell-penetrating peptide dimer according to the present invention, the intrinsic function of the present invention, that is, cell-penetrating properties, will be exerted as it is, without essentially affecting the protein tertiary structure of the peptide dimer according to the present invention. Therefore, as described above, the constitution of the cell-penetrating peptide dimer of the present invention comprising the first and second peptide domains and the peptide linker connected thereto, should be construed as further including an embodiment in which the cell-penetrating peptide dimer is used as it is, or a cell-penetrating peptide dimer essentially consisting of the cell-penetrating peptide dimer by applying conventional modification techniques in the field of protein engineering within a range that does not affect the essential function of the cell-penetrating peptide dimer, in addition to the cell-penetrating peptide dimer consisting of only the first and second peptide domains and the peptide linker connected thereto.

In the present invention, the peptide linker is a linear chain in which carboxyl-terminus of the first peptide domain and the amino-terminus of the second peptide domain are connected to each other, and amino acid residues of natural and/or synthetic origin consisting of 100 or less amino acids are formed by peptide bonds. The peptide linker may remarkably improve cell-penetrating properties of the cell-penetrating peptide dimer according to the present invention by connecting the first peptide domain and the second peptide domain to each other and appropriately inducing the dimer formation of the two domains.

Without being bound by theory, the peptide linker is preferably long enough to provide flexibility to the extent that enables proper protein folding while preventing interfering with each other's activities by steric hindrance, and is also preferably short enough to provide stability (for example, proteolytic stability) within cells.

In an embodiment, the peptide linker has 1 to 100 amino acids in length. For example, the peptide linker has 1, 2, 3, 4 or 5 or more amino acids in length and has 100, 95, 90, 85, 80, 75, 70, 65, 60, 55 or 50 or less amino acids in length. Specifically, the peptide linker may have 1 to 75 amino acids or 5 to 75 amino acids in length, and more specifically, may have 5 to 50 amino acids in length.

In an embodiment, the peptide linker has 5 or more, 10 or more, 15 or more, or 20 or more amino acids in length, and has 50 or less, 40 or less or 30 or less amino acids in length. Specifically, the peptide linker may have 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length.

In the technical field to which the present invention pertains, the peptide linkers are classified into flexible, rigid and in vivo cleavable linkers (see Advanced drug delivery reviews 65.10 (2013): 1357-1369 and Biotechnology advances 33.1 (2015): 155-164), and the like). Among these, the peptide linker of the present invention may be a flexible linker or a rigid linker. In order to provide a novel cell-penetrating peptide dimer, which is the technical subject of the present invention, the present inventors selected peptide linkers recognized as representing flexible linkers and rigid linkers among the peptide linkers in the field of protein engineering, designed a total of 7 kinds of novel cell-penetrating peptide dimers (Examples 1 to 7) (FIG. 5), and confirmed an effect of improving cell-penetrating properties thereof (FIGS. 6 and 7).

In an embodiment, the peptide linker is a flexible linker. In the flexible linker, at least 50%, 60%, 70%, 80%, 90%, 95% or 100% of the amino acids may include specific amino acid residues such as glycine, serine and/or threonine. In an embodiment, the flexible linker may include repeated sequences of specific amino acids. In an embodiment, the flexible linker includes glycine (G) and serine (S). In a more specific embodiment, the flexible linker of the present invention may include an amino acid sequence repeating unit consisting of glycine (G) and serine (S).

The flexible linkers have been widely used in the field of protein engineering due to advantages of being able to impart mobility, flexibility, and interaction of each peptide domain connected to the linker. Among the flexible linkers, a flexible linker characterized by including glycine (G) and serine (S) residues, which are amino acids having a small size, has been typically used (see Advanced drug delivery reviews 65.10 (2013): 1357-1369, Biotechnology advances 33.1 (2015): 155-164, and Protein science 22.2 (2013): 153-167, and the like).

For example, the flexible linker includes 1 to 20 repeating units of G, S, GGS, GSS, GGGS, GGSS, GSSS, GGGGS, GGGSS, GGSSS, GSSSS, GGGGGS, GGGGSS, GGGSSS, GGSSSS, and GSSSSS. In this case, 10 or less, 5 or less, 4 or less, 3 or less, 2 or less, or 1 additional optional amino acid may be added to the amino-terminus and/or carboxyl-terminus of the peptide linker.

The present inventors synthesized cell-penetrating peptide dimers (Examples 1 to 6) using G and S amino acid residues-containing repeated sequences (SEQ ID NOs: 2 to 7), which are known to represent the structural and functional characteristics of the flexible linker in the field of protein engineering (FIG. 5), and experimentally confirmed that the synthesized cell-penetrating peptide dimer had significantly improved cell-penetrating properties compared with those of the existing peptide monomer (Comparative Example 1) and a peptide dimer without a peptide linker (Comparative Example 2) (FIG. 7). Therefore, from the results, a person skilled in the art can reasonably predict that the cell-penetrating peptide dimer using the flexible linker will exhibit an equivalent level of the effect of improving the cell-penetrating properties.

In an embodiment, the peptide linker is a rigid linker. The rigid linker includes 1 to 10 repeating units of EAAAK. In another embodiment, the rigid linker includes 2 to 25 repeating units of XP (wherein X is any amino acid residue, preferably alanine, lysine or glutamic acid). The rigid linker is a peptide linker that has distinct properties from the flexible linker, and has been widely used in the field of protein engineering due to the advantage of maintaining a spacing between each peptide domain to have less interference with independent functions thereof. Among the rigid linkers, a rigid linker consisting of EAAAK repeated sequence are best studied and known to represent structural and functional characteristics of other rigid linkers (see Advanced drug delivery reviews 65.10 (2013): 1357-1369, and Biotechnology advances 33.1 (2015): 155-164, and the like). Thus, the present inventors synthesized a cell-penetrating peptide dimer (Example 7) using the EAAAK repeated sequence (SEQ ID NO: 8) (FIG. 5), and experimentally confirmed that the synthesized cell-penetrating peptide dimer had significantly improved cell-penetrating properties compared with those of the existing peptide monomer (Comparative Example 1) and the peptide dimer without the peptide linker (Comparative Example 2) (FIG. 7). Therefore, from the results, a person skilled in the art can reasonably predict that an effect of improving the cell-penetrating properties for the cell-penetrating peptide dimer using the rigid linker will be exerted at an equivalent level.

In the present invention, the peptide linker includes a peptide linker variant not only having the specified structure, but also performing a biological function equivalent thereto. Herein, the peptide linker variant refers to a peptide having one or more amino acid mutations or modifications compared with the reference peptide linker, for example, may be prepared by substitution, deletion, insertion and/or chemical modification in one or more amino acids in the existing amino acid sequence.

In an embodiment, the peptide linker variant includes conservative amino acid substitutions that do not significantly affect the original function performed by the peptide linker variant. The conservative substitution may include basic amino acids (arginine, lysine and histidine), acidic amino acids (glutamic acid and aspartic acid), polar amino acids (glutamine and asparagine), hydrophobic amino acids (leucine, isoleucine, valine and methionine), aromatic amino acids (phenylalanine, tryptophan, and tyrosine), and small amino acids (glycine, alanine, serine and threonine). In general, amino acid substitutions that do not alter specific activity are known in the art to which the present invention pertains. The most common exchanges include Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly, and other examples of the conservative amino acid substitutions are shown in Table 1 below.

TABLE 1

| Original Amino | Exemplary Residue | Preferred Residue |
|---|---|---|
| Acid | Substitution | Substitution |
| Ala(A) | Val, Leu, Ile | Val |
| Arg(R) | Lys, Gln, Asn | Lys |
| Asn(N) | Gln, His, Asp, Lys, Arg | Gln |
| Asp(D) | Glu, Asn | Glu |
| Cys(C) | Ser, Ala | Ser |
| Gln(Q) | Asn, Glu | Asn |
| Glu(E) | Asp, Gln | Asp |
| Gly(G) | Ala | Ala |
| His(H) | Asn, Gln, Lys, Arg | Arg |
| Ile(I) | Leu, Val, Met, Ala, Phe | Leu |
| Leu(L) | Ile, Val, Met, Ala, Phe | Ile |
| Lys(K) | Arg, Gln, Asn | Arg |
| Met(M) | Leu, Phe, Ile | Leu |
| Phe(F) | Leu, Val, Ile, Ala, Tyr | Tyr |
| Pro(P) | Ala | Ala |
| Ser(S) | Thr | Thr |
| Thr(T) | Ser | Ser |
| Trp(W) | Tyr, Phe | Tyr |
| Tyr(Y) | Trp, Phe, Thr, Ser | Phe |
| Val(V) | Ile, Leu, Met, Phe, Ala | Leu |

In an embodiment, the peptide linker is consisting of the amino acid sequence of any one of SEQ ID NOs: 2 to 8. In an embodiment, the peptide linker is a peptide linker variant having an amino acid sequence with at least 80%, 85%, 90%, 95%, 96%, 97%, 98% or 99% homology to the amino acid sequence of any one of SEQ ID NOs: 2 to 8. In an embodiment, the peptide linker is a conservative amino acid substitute for the amino acid sequence of any one of SEQ ID NOs: 2 to 8.

The peptide linker of the present invention may be encoded by a nucleic acid molecule and thus may be expressed as a recombinant protein. In addition, the peptide linker may be expressed as the recombinant protein by linking a first peptide domain and a second peptide domain with a peptide bond.

In an embodiment, the cell-penetrating peptide dimer of the present invention is a fusion protein. In the present invention, the fusion protein refers to a protein in a form in which amino acid sequences of different origins are combined into one polypeptide chain by in-frame combination of nucleotide sequences encoding the amino acid sequences, and includes an internal fusion in which a sequence of a different origin is inserted into a polypeptide chain, as well as being fused to one of ends of the polypeptide chain. The fusion protein may be a recombinant protein prepared from any one selected from the group consisting of E. coli, yeast, insect cells, and mammalian cells by a gene recombination method.

In an embodiment, a cargo may be conjugated to the cell-penetrating peptide dimer of the present invention. In the present invention, “conjugation” refers to any form of covalent or non-covalent linkage, and includes direct genetic or chemical fusion, coupling through a crosslinking agent, and non-covalent interaction, such as interaction through van der Waals force.

In Examples of the present invention, the present inventors prepared a cell-penetrating peptide dimer protein including a peptide linker having various types and lengths (FIG. 5), and confirmed that the protein had remarkably improved cell-penetrating properties compared with those of not only the conventional peptide consisting of the amino acid of SEQ ID NO: 1, but also a peptide dimer that did not contain a linker at all (FIGS. 6 and 7).

As described above, the peptide linker is typically divided into a flexible linker and a rigid linker. In FIG. 7, Examples 1 to 6 show experimental results using a GGSSS repeated amino acid sequence (5 to 50 in length) representing the flexible linker, and Example 7 shows experimental results using an EAAAK repeated amino acid sequence (20 in length) representing the rigid linker. Examples 1 to 6 show experimental results when the same flexible linker is used but each linker has different lengths, and Example 7 shows experimental results when the flexible linker is changed to the rigid linker. It may be confirmed that even if the length of each linker is different (Examples 1 to 6), and the type of linker is changed from the flexible linker to the rigid linker (Example 7), utility of the invention, that is, an effect of improving cell-penetrating properties, compared with those of Comparative peptides, has been sufficiently achieved in common. In particular, compared with the peptide of Comparative Example 2 in which the peptide domain of SEQ ID NO: 1 is linked without using the peptide linker, the effect of improving cell-penetrating properties has been sufficiently achieved in Examples 1 to 7 using various peptide linkers, which well reflects the core technical idea of the present invention, i.e., a 30Kc19α dimer configuration linked through the “peptide linker”.

Method for Preparing Cell-Penetrating Peptide Dimer

In an aspect, the present invention provides a method for preparing the cell-penetrating peptide dimer described above. To this end, the present invention provides a nucleic acid encoding the cell-penetrating peptide dimer described above, a recombinant vector containing the nucleic acid, a host cell transformed with the recombinant vector, and a method for preparing a cell-penetrating peptide dimer using the host cell.

In an aspect, the present invention provides a nucleic acid encoding the cell-penetrating peptide dimer described above. Those of ordinary skill in the art will appreciate that in consideration of codon degeneracy or preferred codon in an organism to which the cell-penetrating peptide dimer is intended to be expressed, the nucleic acid may be variously modified in a coding region within a range in which the amino acid sequence of the cell-penetrating peptide dimer expressed from the coding region is not altered, may be subjected to various modifications or alterations even in a region except the coding region, provided that it does not affect the expression of the gene, and such modified genes are also included in the scope of the present invention. In other words, as long as the nucleic acid of the present invention encodes a protein having an activity equivalent thereto, one or more nucleic acid bases may be mutated by substitution, deletion, insertion, or a combination thereof, and these are also included in the scope of the present invention.

In an aspect, the present invention provides a recombinant vector including the nucleic acid described above. In the present invention, the recombinant vector is a vehicle capable of introducing a foreign nucleic acid into a host cell, transforming the host cell, and promoting expression of the introduced nucleic acid, which is used with the same meaning as commonly used in the technical field to which the present invention pertains.

In an embodiment, the recombinant vector is a plasmid vector, a cosmid vector, a viral vector or an artificial chromosomal vector. The plasmid vector is a DNA molecule capable of easily accommodating foreign DNA and being easily introduced into the host cell, which is used with the same meaning as commonly used in the technical field to which the present invention pertains. A typical plasmid vector has a structure including (a) the origin of replication for efficient replication to contain hundreds of plasmid vectors per a host cell, (b) a selectable marker that allows the host cell transformed with a plasmid vector to be selected, and (c) a restriction enzyme cleavage site into which foreign DNA fragments are capable of being inserted. Even if an appropriate restriction enzyme cleavage site does not exist, the vector and foreign DNA are able to be easily ligated when using a synthetic oligonucleotide adapter or linker according to a conventional method. Non-limiting examples of the plasmid vector may include, but are not limited to, pKK plasmid (Clonetech), pUC plasmid, pET plasmid (Novagen, Inc., Madison, Wis.), pRSET or pREP plasmid (Invitrogen, San Diego, Calif.), or pMAL plasmid (New England Biolabs, Beverly, Mass.).

In an embodiment, the viral vector may be a DNA viral vector or an RNA viral vector, and may be a bacteriophage, an animal virus, or a plant virus. For example, the viral vector may be vaccinia virus, adenovirus, adeno-associated virus (AAV), lentivirus, retrovirus, or herpes virus, but is not limited thereto.

In the present invention, the recombinant vector includes a nucleic acid encoding a first peptide domain consisting of the amino acid sequence of SEQ ID NO: 1; a nucleic acid encoding a second peptide domain consisting of the amino acid sequence of SEQ ID NO: 1; and a nucleic acid encoding a peptide linker connecting the 3′end of the nucleic acid encoding the first peptide domain and the 5′end of the nucleic acid encoding the second peptide domain to each other.

In an embodiment, the recombinant vector includes a promoter operably linked to a nucleic acid encoding the cell-penetrating peptide dimer. In the present invention, the term “operably linked” means that a specific nucleic acid is linked to another nucleic acid so as to exert function thereof. In other words, the fact that a gene encoding the specific protein or peptide is operably linked to a promoter means that the corresponding gene is capable of being transcribed into mRNA by the promoter and translated into a protein or peptide.

In an embodiment, the recombinant vector further includes a nucleic acid encoding a cargo which is a peptide or protein, wherein the nucleic acid encoding the cargo is linked to the 5′end of the nucleic acid encoding the first peptide domain or the 3′end of the nucleic acid encoding the second peptide domain. The meaning of the cargo which is the peptide or protein is the same as described above.

In an aspect, the present invention provides a host cell transformed with the recombinant vector described above. In the present invention, the term “transformation” refers to a process of introducing a gene into a host cell to be capable of being expressed in the host cell, wherein the transformed gene includes any gene without limitation, either inserted into chromosome of the host cell or presented in the outside of chromosome as long as it is capable of being expressed in the host cell. In addition, the gene includes DNA and RNA as nucleic acids capable of encoding a polypeptide. Any type of gene is usable without limitation as long as the gene is capable of being introduced and expressed into the host cell. A method for transforming by introducing the recombinant vector of the present invention into the host cell includes methods known in the technical field to which the present invention pertains, using a recombinant vector containing the DNA of the present invention, such as transient transfection, microinjection, transduction, cell fusion, calcium phosphate precipitation, liposome-mediated transfection, DEAE dextran-mediated transfection, polybrene-mediated transfection, electroporation, and the like, but the method is not limited thereto.

In an embodiment, the host cell may be prokaryotic cells such as E. coli; fungi such as yeast; plant cells; or mammalian cells such as an insect cell or a plant cell, and may be a cell line derived from the cell. Non-limiting examples of the cell line may include, but are not limited to, CHO, HeLa, COST, COPS, HEK293, HEK293T, HepG2, CV1, BHK, TM4, VERO-76, MDCK, W138, and the like. A person skilled in the art may appropriately select the type of host cell suitable for preparation of the recombinant protein.

In an aspect, the present invention provides a method for preparing a cell-penetrating peptide dimer using a host cell transformed with the recombinant vector as described above.

In an embodiment, the method for preparing a cell-penetrating peptide dimer includes transforming the recombinant vector described above into the recombinant host cell described above; culturing the host cell; inducing expression of a cell-penetrating peptide dimer from the cultured host cell; and recovering the expressed cell-penetrating peptide. The method for culturing the host cell, the method for inducing expression of the protein, and the method for recovering the expressed protein that are suitable for preparing the recombinant fusion protein are known in the technical field to which the present invention pertains, and a person skilled in the art can appropriately select a method for effectively expressing the cell-penetrating peptide dimer according to the present invention using the above-described vector and host cell.

Cargo Delivery System Using Cell-Penetrating Peptide Dimer

In an aspect, the present invention provides a cargo delivery system including the cell-penetrating peptide dimer described above and a cargo conjugated thereto.

In the present invention, “cargo” refers to all substances that are conjugated to the peptide dimer according to the present invention to be movable into cells, and for example, may be any material that is desired to increase cell-penetration efficiency, specifically, an effective material of a drug, a cosmetic product, or a health food, and more specifically, a material that is not easily transported into cells through a general route. Drugs delivered into cells may further include drug delivery systems such as liposomes, micelles, magnetic particles, or quantum dots.

In an embodiment, the cargo is a peptide, a protein, a nucleic acid, a lipid, a glycolipid, a carbohydrate, a mineral, a nanoparticle, a virus, a contrast material, a chemical substance, or a combination thereof.

When the cargo is the peptide or protein, the cargo may include, but are not limited to, hormones, hormone analogs, cytokines, neurotransmitter peptides, vaccines, antibodies, antibody fragments, enzymes, enzyme inhibitors, soluble receptors, signal transduction proteins, transcription factors, co-activators, transcription inhibitory proteins, mitochondrial proteins. In this case, the cargo may be conjugated to the peptide dimer in the form of the cargo and peptide dimer fusion protein by binding the DNA expressing the peptide dimer according to the present invention to the DNA expressing the cargo peptide and then expressing the bound DNAs at once.

When the cargo is a nucleic acid, the cargo may be a naturally occurring or artificial DNA or RNA molecule, and may be single-stranded or double-stranded. The number of nucleic acid molecules may be one or more, and these nucleic acid molecules may be the same type (for example, having the same nucleotide sequence) or may be of different types of nucleic acid molecules. Specifically, the nucleic acid includes, but is limited to, one or more of cDNA, decoy DNA, cfDNA, ctDNA, RNA, siRNA, miRNA, shRNA, stRNA, snoRNA, snRNA, PNA, antisense oligomers, plasmids, and other modified nucleic acids.

The contrast material refers to any material used for imaging in vivo structures or fluids in medical imaging. The contrast material includes, but is not limited to, radiopaque contrast materials, paramagnetic contrast materials, superparamagnetic contrast materials, CT contrast materials, or other contrast materials.

In an embodiment, the cargo is covalently linked to a cell-penetrating peptide dimer.

In an embodiment, the cargo is covalently linked to the amino-terminus of the first peptide domain or the carboxyl-terminus of the second peptide domain of the cell-penetrating peptide dimer.

The present inventors prepared a cell-penetrating peptide dimer protein (FIG. 5), and confirmed that the protein had significantly improved cell-penetrating properties compared with those of a peptide dimer protein that did not contain the linker at all as well as a conventional monomer (FIGS. 6 and 7).

In Examples of the present invention, the present inventors prepared a cargo delivery system in which a cargo protein (GFP) is connected to a cell-penetrating peptide dimer protein including peptide linkers each having various types and lengths (FIG. 5), and confirmed that the cargo delivery system had remarkably improved cell-penetrating properties compared with those of the dimeric cargo delivery system that did not contain the linker at all as well as the conventional monomer cargo delivery system (FIGS. 6 and 7).

That is, the delivery system according to the present invention is characterized by having significantly improved cell delivery efficiency compared with that of the conventional cell-penetrating peptide monomer cargo delivery system.

Without being bound by theory, the remarkable cellular penetration effect of the dimer delivery system newly confirmed by the present inventors may be explained as follows.

In view of the composition of the binding, it is assumed that the conventional cell-penetrating peptide monomer cargo delivery system has a steric hindrance since relatively high molecular weight cargos in the conjugated forms float in vitro or in vivo space and then should form the dimer (FIG. 2A). On the other hand, it is assumed that the cargo delivery system according to the present invention has a higher probability of forming the dimer since each 30Kc19α domain is able to move within the range of the peptide linker and bind to each other regardless of the in vitro or in vivo space (FIG. 2B).

In view of the formation of peptide dimer in vitro, it is estimated that the conventional 30Kc19α cell-penetrating peptide monomer cargo delivery system has a lower probability of forming the dimer at a concentration used in vivo, and thus the cell-penetration effect may be poor (FIG. 3A). On the other hand, it is estimated that the cargo delivery system according to the present invention exhibits a high probability of forming the dimer even in an in vivo environment, thereby remarkably improving the cell-penetration effect (FIG. 3B).

In view of molecular weight due to dimer formation, it is estimated that when forming a dimer in the conventional 30Kc19α cell-penetrating peptide monomer cargo delivery system, since there are two cargo materials bound to the dimer, a molecular weight is relatively excessive, and thus the cell-penetration efficiency may be poor (FIG. 4A). On the other hand, it is estimated that in the case of the cargo delivery system according to the present invention, since one cargo material is linked to the 30Kc19α domain dimer, the cell-penetration effect is remarkably improved (FIG. 4B).

Therefore, the cargo delivery system using the 30Kc19α cell-penetrating peptide dimer of the present invention has significantly improved characteristics compared with those of the cargo system using the conventional 30Kc19α cell-penetrating peptide monomer in the route of administration in vivo or clinical conditions and the resulting pharmacokinetics.

Use of Cargo Delivery System Using Cell-Penetrating Peptide Dimer

In an aspect, the cargo delivery system described above may be used in pharmaceuticals, cosmetics or food. In an embodiment, the present invention provides a pharmaceutical composition, cosmetic composition, or food composition including the cargo delivery system described above.

The composition may contain the above-described cell-penetrating peptide dimer in an amount suitable for pharmaceuticals, cosmetics, or food. The composition may be applied to all animals including humans, dogs, chickens, pigs, cows, sheep, guinea pigs or monkeys.

The pharmaceutical composition may be administered by oral, rectal, transdermal, intravenous, intramuscular, intraperitoneal, intramedullary, intrathecal or subcutaneous route. A formulation for oral administration may be tablets, pills, soft or hard capsules, granules, powders, liquids, or emulsions, but is not limited thereto. A formulation for parenteral administration may be injections, drops, lotions, ointments, gels, creams, suspensions, emulsions, suppositories, patches, or sprays, but is not limited thereto. The pharmaceutical composition may include additives such as diluents, excipients, lubricants, binders, disintegrants, buffers, dispersants, surfactants, colorants, flavors, or sweeteners, and the like, if necessary.

The cosmetic composition may be provided in any formulation suitable for topical application. For example, the cosmetic composition may be provided in the form of a solution, an emulsion obtained by dispersing an oil phase in an aqueous phase, an emulsion obtained by dispersing an aqueous phase in an oil phase, a suspension, a solid, a gel, a powder, a paste, a foam, or an aerosol. These formulations may be prepared according to conventional methods in the art. The cosmetic composition may preferably contain other ingredients that are capable of providing a synergistic effect to the main effect, within a range that does not impair the main effect. Further, the cosmetic composition may further include a moisturizer, an emollient, a surfactant, an ultraviolet absorber, a preservative, a disinfectant, an antioxidant, a pH adjuster, an organic or inorganic pigment, a fragrance, a cooling agent or a limiting agent. The blending amount of the above-described ingredients may be easily selected by a person skilled in the art within a range that does not impair the object and effect of the present invention.

The formulation of the food composition is not particularly limited, but may be formulated as, for example, tablets, granules, powders, liquids, solid preparations, and the like. In each formulation, a person skilled in the art may appropriately select and blend ingredients commonly used in the corresponding field, in addition to active ingredients, without difficulty depending on the formulation or purpose of use, wherein a synergistic effect may occur when applied simultaneously with other raw materials.

### BRIEF DESCRIPTION OF THE DRAWINGS

FIG. 1 shows a cell-penetrating peptide dimer of the present invention;

FIGS. 2A, 2B, 3A, 3B, 4A, and 4B visually explain the improved cell-penetration effect of the cell-penetrating peptide dimer of the present invention;

FIG. 5 is an image showing results of preparing the cell-penetrating peptide dimer of the present invention;

FIG. 6 is an image confirming an intracellular cell-penetration ability of the cargo-conjugated cell-penetrating peptide dimer of the present invention by immunocytochemistry; and

FIG. 7 shows a graph showing comparison of cell-penetration abilities of the cargo-conjugated cell-penetrating peptide dimers of the present invention using various linkers.

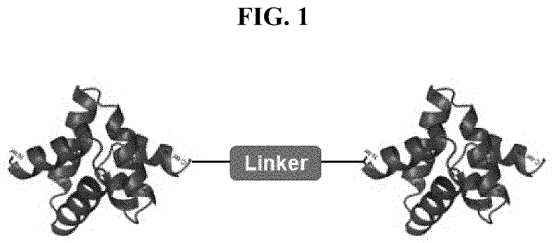

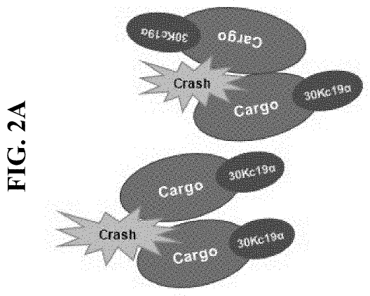

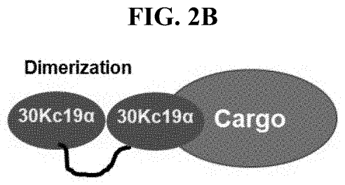

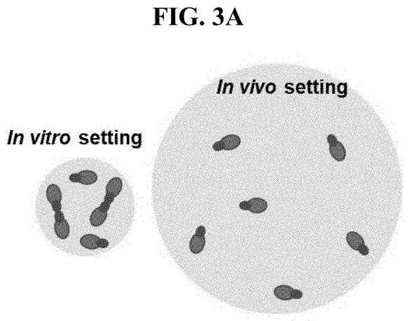

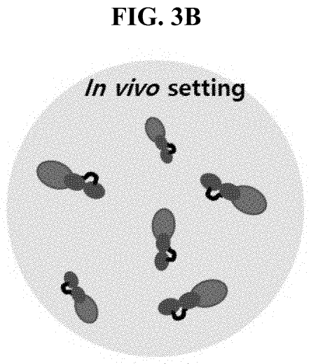

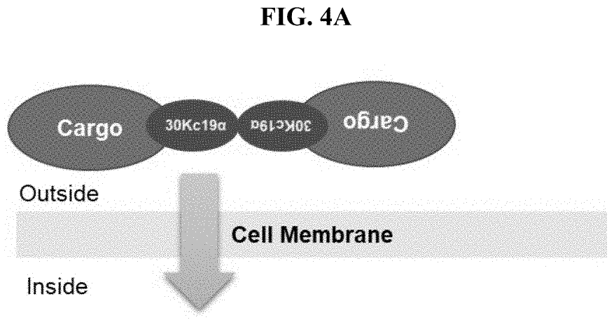

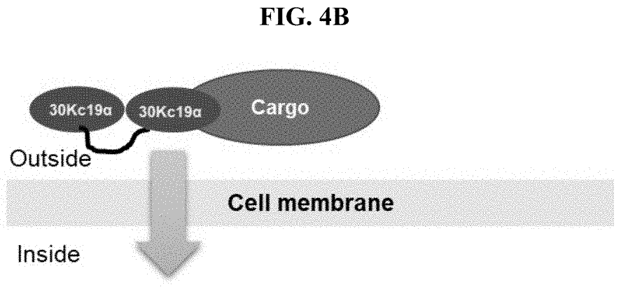

### DETAILED DESCRIPTION OF EXEMPLARY EMBODIMENTS

Hereinafter, the present invention will be described in more detail by Examples. However, these Examples of the present invention are provided to facilitate understanding of the invention, and the scope of the invention to be protected is not limited by the following Examples.

<Experimental Materials and Methods>

1. Construction of Plasmid

As a plasmid for production of a recombinant protein, a pET-23a vector (Novagen, Madison, Wis., USA), which is advantageous for expression in E. coli and purification of His tag protein, was purchased and used. The entire 30Kc19α genes containing a linker or 30Kc19α were synthesized by Geneart. During the synthesis, codon optimization was performed according to the E. coli strain.

The TurboGFP-30Kc19α gene sequence (SEQ ID NO: 10) in which the TurboGFP gene sequence was fused to the upstream of the 30Kc19α gene sequence (SEQ ID NO: 9) was inserted using the BamHI/Xhol restriction enzyme site in the multiple cloning site (MCS) of the pET-23a vector. The synthesized genes were inserted into the pET-23a vector containing TurboGFP using the EcoRI/Xhol restriction enzyme site in the sequence.

Specifically, first, each vector and insert added with EcoRI/Xhol enzyme (NEB) and a custom buffer were cultured for 18 hours in a heat block at 37° C. Next, the insert and vector were subjected to electrophoresis on an agarose gel, and then each insert and vector were extracted from the gel using AccuPrep® PCR/Gel DNA Purification Kit (Bioneer). The extracted insert and vector were mixed in a ratio of 3:1, and then T4 DNA ligase (Cat No. M0202M from NEB) was added and cultured for 18 hours. The resulting plasmid was transformed into DH5a competent cells (Cat. No. RH617-J80 from RBC), and then only ampicillin resistant colonies were selected and cultured in 2 ml of LB broth media (0.1% ampicillin) for 12 hours. The plasmid was extracted from the cell pellets using AccuPrep® Nano-Plus Plasmid Extraction Kit (Bioneer), and the final nucleotide sequence was confirmed.

2. Preparation and Purification of Recombinant Protein

The plasmid extracted according to the above-described procedure was transformed into BL21 competent cells, put into LB broth medium, and cultured in a shaking incubator. The product was treated with IPTG 1 mM, and cultured for 4 hours. Cell lysates were obtained using a centrifuge and lysed using a sonicator. Then, the protein was purified using FPLC (GE Healthcare), followed by dialysis, and stored. As purification buffers, lysis buffer (20 mM Tris-HCl, 0.5 M NaCl, 20 mM imidazole, pH 8.0), washing buffer (20 mM Tris-HCl, 0.5 M NaCl, 50 mM imidazole, pH 8.0), elution buffer (20 mM Tris-HCl, 0.5 M NaCl, 350 mM imidazole, pH 8.0), and dialysis buffer (20 mM Tris-HCl buffer, pH 8.0) were used.

As a result, recombinant proteins (Examples 1 to 7) having the structure of N′-GFP-30Kc19α-linker-30Kc19α-C′ were obtained. All of the GFP-30Kc19α contained in the recombinant proteins of Examples 1 to 7 had the amino acid sequence of SEQ ID NO: 1, and the linkers had the amino acid sequences shown in the following table. Here, the linkers L5, L10, L20, L30, L40 and L50 were flexible linkers having 5 to 50 amino acids in length (GGSSS repeated sequence), and Rigid 20 is a rigid linker having 20 amino acids in length (EAAAK repeated sequence).

| Example | Linker | Amino Acid Sequence | SEQ ID NO. |
|---|---|---|---|
| Example 1 | L5 | GGSSS | SEQ ID NO. 2 |
| Example 2 | L10 | GGSSSGGSSS | SEQ ID NO. 3 |
| Example 3 | L20 | GGSSSGGSSSGGSSSGGSSS | SEQ ID NO. 4 |
| Example 4 | L30 | GGSSSGGSSSGGSSSGGSSSGGSSSGGSSS | SEQ ID NO. 5 |
| Example 5 | L40 | GGSSSGGSSSGGSSSGGSSSGGSSSGGSSS | SEQ ID NO. 6 |
| | | GGSSSGGSSS | |
| Example 6 | L50 | GGSSSGGSSSGGSSSGGSSSGGSSSGGSSS | SEQ ID NO. 7 |
| | | GGSSSGGSSSGGSSSGGSSS | |
| Example 7 | Rigid 20 | EAAAKEAAAKEAAAKEAAAK | SEQ ID NO. 8 |

As Comparative Peptides, a recombinant protein having a structure of N′-GFP-30Kc19α-C′ (Comparative Example 1) and a recombinant protein having a structure of N′-GFP-30Kc19α-30Kc19α-C′ (Comparative Example 2) were also prepared. The GFP-30Kc19α contained in Comparative Examples 1 and 2 all had the amino acid sequence of SEQ ID NO: 1.

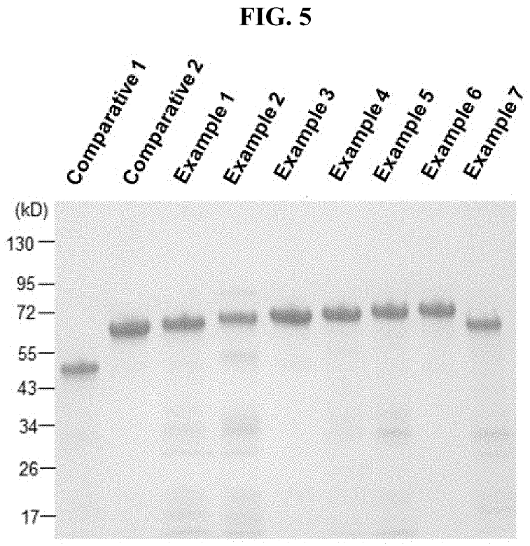

The prepared protein was confirmed using SDS-PAGE. The prepared protein having an amount of 3 μg was mixed with 4× Laemmli sample buffer (Bio-Rad, Cat. No. 1610747) and heated for 5 minutes using a 95° C. heat block. Each sample was loaded onto a 4-15% Mini-PROTEAN TGX stain-free gel (Bio-Rad, Cat. No. 456-1085), then stained with Coomassie Brilliant Blue G-250 (ThermoFisher Scientific, Cat. No. 20279), and observed. Results of the prepared proteins were shown in FIG. 5.

### EXPERIMENTAL EXAMPLE

### Experimental Example 1: Confirmation of Cell-Penetration Ability of Cell-Penetrating Peptide Dimer Using Immunocytochemistry

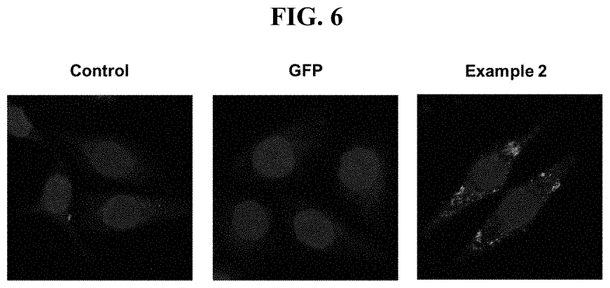

For immunocytochemistry experiments, HeLa cells and the GFP-conjugated cell-penetrating peptide dimer (Example 2) according to the present invention were incubated for 4 hours, and then strongly washed three times with PBS. The cells were fixed by treatment with 4% paraformaldehyde for 20 minutes, followed by incubation with PBS containing 0.25% Triton X-100 for 10 minutes to perform permeabilization. The fixed cells were blocked with 0.1% PBS-T containing 3% BSA for 1 hour. Then, the cells were incubated at 4 t for 16 hours with an anti-Rab-7 antibody (anti-mouse) (Cat. No. ab50533; Abcam, USA) and an anti-turboGFP antibody (anti-rabbit) (Invitrogen, Cat. No. PAS-22688). After washing three times with PBS for 10 minutes each, the cells were treated for 1 hour with secondary antibodies, that is, an anti-rabbit antibody (Invitrogen, Cat. No. A32731) and an anti-mouse antibody (Invitrogen, Cat. No. A32744) each diluted to 1:2000. After washing again 5 times with PBS for 10 minutes each, the cell nuclei were stained with Hoechst 33342 for 10 minutes. After washing with PBS for 10 minutes, intracellular fluorescence was observed using a confocal laser microscope (Leica, Germany), and images were taken using software (Leica). Results are shown in FIG. 6.

In FIG. 6, blue fluorescence indicates cell nuclei, red fluorescence indicates intracellular distribution of the endosome marker Rab7, and green fluorescence indicates intracellular distribution of GFP. In the control group to which the peptide was not added to the HeLa cells (Control) and the experimental group treated with the GFP protein alone (GFP), the green fluorescence did not show in the cells, whereas, in the experimental group treated with the GFP-conjugated cell-penetrating peptide dimer (Example 2), the green fluorescence was clearly observed in the cells. That is, it was confirmed that the cell-penetrating peptide dimer according to the present invention had an effective cell-penetration ability (FIG. 6).

### Experimental Example 2: Confirmation of Cell-Penetration Ability of Cell-Penetrating Peptide Dimers According to Various Linkers

HeLa cells were seeded in a 96-well plate to have a confluency of 70% and stabilized for 24 hours. The GFP-conjugated peptides of Examples 1 to 7 prepared in the above-described Examples were added to the medium at a final concentration of 1 μM. After 1 hour, the peptides were washed three times with PBS, and the GFP fluorescence intensity was measured using a plate reader.

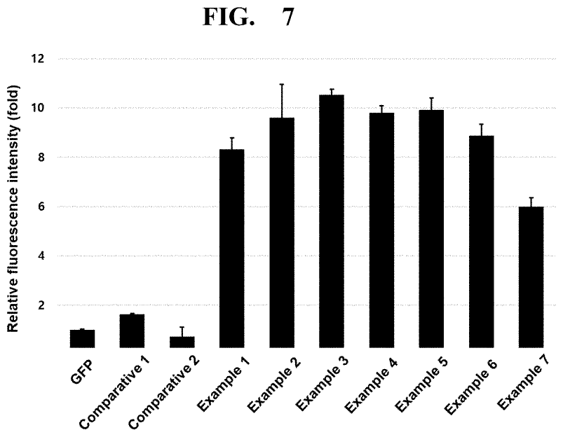

As a result, it was confirmed that the cell-penetrating peptide dimer according to the present invention had significantly improved cell-penetrating properties compared with those of not only the peptide of Comparative Example 1, which was the cell-penetrating peptide monomer but also the peptide of Comparative Example 2, which was the peptide dimer without using the linker. Furthermore, it was confirmed that all of the cell-penetrating peptide dimers of the present invention using linkers having various lengths and amino acid configurations had remarkably excellent cell-penetrating properties compared with those of Comparative Examples (FIG. 7).

The cell-penetrating peptide dimer according to the present invention has remarkably excellent cell-penetrating properties compared with those of the 30Kc19α peptide monomer, thereby being usefully employed as a cargo delivery system.

Although some Examples of the present invention have been shown and described, those skilled in the art having ordinary skill in the art to which the present invention pertains will appreciate that the present embodiments can be modified without departing from the principles or spirit of the present invention. The scope of the present invention will be determined by the appended claims and equivalents thereof.

What is claimed is:

1. A cell-penetrating peptide dimer comprising:
a first peptide domain consisting of the amino acid sequence of SEQ ID NO: 1;
a second peptide domain consisting of the amino acid sequence of SEQ ID NO: 1; and
a peptide linker connecting the first and second peptide domains,
wherein the cell-penetrating peptide dimer is a fusion protein, and the peptide linker connects a carboxyl-terminus of the first peptide domain and an amino-terminus of the second peptide domain to each other, and consists of 100 or less amino acids,
wherein the peptide linker is a flexible linker or a rigid linker.

2. The cell-penetrating peptide dimer according to claim 1, wherein the peptide linker is the flexible linker including glycine (G) and serine (S).

3. The cell-penetrating peptide dimer according to claim 2, wherein the peptide linker is the flexible linker comprising an amino acid sequence repeating unit consisting of glycine (G) and serine (S).

4. The cell-penetrating peptide dimer according to claim 1, wherein the peptide linker is a rigid linker comprising repeating units of amino acid sequence EAAAK.

5. The cell-penetrating peptide dimer according to claim 1, wherein the peptide linker consists of the amino acid sequence of any one of SEQ ID NOs: 2 to 8.

6. The cell-penetrating peptide dimer according to claim 1, wherein a cargo is conjugatable.

7. A nucleic acid encoding the cell-penetrating peptide dimer according to claim 1.

8. The nucleic acid according to claim 7, wherein the nucleic acid encoding the peptide linker connects the 3′end of the nucleic acid encoding the first peptide domain and the 5′ end of the nucleic acid encoding the second peptide domain to each other.

9. The nucleic acid according to claim 8, which further comprises a nucleic acid encoding a cargo that is a peptide or a protein, wherein the nucleic acid encoding the cargo is connected to the 5′end of the nucleic acid encoding the first peptide domain or the 3′ end of the nucleic acid encoding the second peptide domain.

10. A cargo delivery system comprising the cell-penetrating peptide dimer according to claim 1 and a cargo conjugated to the dimer.

11. The cargo delivery system according to claim 10, wherein the cargo is a peptide, a protein, a nucleic acid, a lipid, a glycolipid, a carbohydrate, a mineral, a nanoparticle, a virus, a contrast material, a chemical substance, or a combination thereof.

12. The cargo delivery system according to claim 11, wherein the cargo is covalently linked to the cell-penetrating peptide dimer.

13. The cargo delivery system according to claim 12, wherein the cargo is covalently linked to an amino-terminus of the first peptide domain or a carboxyl-terminus of the second peptide domain of the cell-penetrating peptide dimer.


SEQUENCE LISTING (10 sequences)

SEQ ID NO: 1 - protein, 88 aa, unidentified
  REGION 1..88: note = Artificial
ADSDVPNDILEEQLYNSVVVADYDSAVEKSKHLYEEKKSEVITNVVNKLIRNNKMNCMEYAYQLWLQGSKDIVRDCFPVEFRLIFAEN

SEQ ID NO: 2 - protein, 5 aa, unidentified
  REGION 1..5: note = Artificial
GGSSS

SEQ ID NO: 3 - protein, 10 aa, unidentified
  REGION 1..10: note = Artificial
GGSSSGGSSS

SEQ ID NO: 4 - protein, 20 aa, unidentified
  REGION 1..20: note = Artificial
GGSSSGGSSSGGSSSGGSSS

SEQ ID NO: 5 - protein, 30 aa, unidentified
  REGION 1..30: note = Artificial
GGSSSGGSSSGGSSSGGSSSGGSSSGGSSS

SEQ ID NO: 6 - protein, 40 aa, unidentified
  REGION 1..40: note = Artificial
GGSSSGGSSSGGSSSGGSSSGGSSSGGSSSGGSSSGGSSS

SEQ ID NO: 7 - protein, 50 aa, unidentified
  REGION 1..50: note = Artificial
GGSSSGGSSSGGSSSGGSSSGGSSSGGSSSGGSSSGGSSSGGSSSGGSSS

SEQ ID NO: 8 - protein, 20 aa, unidentified
  REGION 1..20: note = Artificial
EAAAKEAAAKEAAAKEAAAK

SEQ ID NO: 9 - DNA, 264 bp, unidentified
  misc_feature 1..264: note = Artificial
  source 1..264: mol_type = other DNA
gcagattccgacgtccctaacgacattctggaggagcagctttacaatagcgtcgtcgtcgccgattacgacagtgcggttgaaaagagcaagcatttatacgaggagaagaagagcgaagtcatcacaaatgtcgtgaacaaactgatacgaaacaacaagatgaactgcatggagtacgcctatcaactttggctccagggctccaaggacatcgtccgggattgtttcccagttgagttcagacttatcttcgccgaaaac

SEQ ID NO: 10 - DNA, 981 bp, unidentified
  misc_feature 1..981: note = Artificial
  source 1..981: mol_type = other DNA
ggatccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaagttgaattcgcagattccgacgtccctaacgacattctggaggagcagctttacaatagcgtcgtcgtcgccgattacgacagtgcggttgaaaagagcaagcatttatacgaggagaagaagagcgaagtcatcacaaatgtcgtgaacaaactgatacgaaacaacaagatgaactgcatggagtacgcctatcaactttggctccagggctccaaggacatcgtccgggattgtttcccagttgagttcagacttatcttcgccgaaaacctcgag